(19) United States
(12) Patent Application Publication
(10) Pub. No.: US 2023/0218178 A1
(43) Pub. Date: Jul. 13, 2023

(54) CONSTRUCTION METHOD AND APPLICATION OF DIGITAL HUMAN CARDIOVASCULAR SYSTEM BASED ON HEMODYNAMICS

(71) Applicant: NANJING UNIVERSITY, Jiangsu (CN)

(72) Inventors: Fei XU, Jiangsu (CN); Hengtian ZHU, Jiangsu (CN); Ye CHEN, Jiangsu (CN); Liuwei ZHAN, Jiangsu (CN); Biao XU, Jiangsu (CN); Qing DAI, Jiangsu (CN)

(73) Assignee: NANJING UNIVERSITY, Jiangsu (CN)

(21) Appl. No.: 17/795,881

(22) PCT Filed: Jul. 6, 2021
(86) PCT No.: PCT/CN2021/106795

§ 371 Date: Jul. 28, 2022

(30) Foreign Application Priority Data
May 26, 2021 (CN) 202110570819.1

(51) Int. Cl. A61B 5/021; A61B 5/024

(52) U.S. Cl. CPC A61B 5/02108; A61B 5/02416

(57) ABSTRACT

A construction method and an application of a digital human cardiovascular system based on hemodynamics are provided, the construction method comprising: partitioning various parts of a human body according to a distributed hemodynamic monitoring system, and determining granularity of a digital human model system; constructing an arterial blood flow transmission equation based on an elastic tube model for arteries, and defining physiological significance for characteristic values of the equation; inputting signals, characteristic values and hemodynamic parameters acquired by the distributed hemodynamic monitoring system into the digital human model system; and obtaining a dynamic and distributed digital human cardiovascular system through finite element calculation. An individual dynamic digital human model based on hemodynamics is constructed through the distributed characteristic values of various parts of the human body and the calculated hemodynamic parameters. The model can dynamically record and display cardiovascular states of various parts of the human body in real time.

### TECHNICAL FIELD

The present invention relates to a construction method of a digital human system, in particular to a construction method and an application of a digital human cardiovascular system based on hemodynamics.

### BACKGROUND TECHNOLOGY

Hemodynamic parameters are closely related to cardiovascular diseases and their extraction is relatively blind. The non-invasive hemodynamic monitoring technology is a hemodynamic monitoring technology including pulse wave detection, electrocardiogram signal detection and phonocardiogram signal detection. The waveform characteristics (such as morphology, intensity, speed and rhythm) exhibited by various signals can reflect many physiological and pathological characteristics of the human cardiovascular system, such as arrhythmia, mitral valve lesion, aortic valve lesion, hypertension, pulmonary hypertension, and heart failure. The non-invasive hemodynamic monitoring technology has advantages such as real-time detection, non-invasive monitor and convenience, and is suitable for cardiovascular health monitoring in multiple scenarios including clinical treatment, daily health management and exercise management.

However, most of the existing products and research works only focus on the hemodynamic characteristics of a certain segment of the arterial system (such as carotid artery, brachial artery, radial artery, and lower limb artery), and the detection and research on the whole arterial system are lacking. In addition, the health states of arteries in different parts of the human body vary, as do the hemodynamic states of different arteries. For example, the blood pressure of the left and right upper limbs of the human body may be different, and the blood pressure of the upper and lower limbs of the human body may also be different. Pathological changes of different parts and properties of the human cardiovascular system have different effects on the waveform characteristics of the hemodynamic signal. Existing non-invasive hemodynamic monitoring technologies are limited to researches on the hemodynamic characteristics of certain segmental arteries, and detection and research on the whole arterial system are relatively lacking.

Digital human is a highly integrated technology of medical science and technology, information science, life science, artificial intelligence, system science, computational science and computer technology today. Nowadays, digital human technology stays on the 3D tomography digital human body based on medical images, and plays an auxiliary role in medical education. The digital physical human model has not yet been constructed by the physiological parameters such as the human cardiovascular system.

### SUMMARY OF THE INVENTION

Objective: the present invention is intended to provide a construction method and an application of a digital human cardiovascular system based on hemodynamics and a systemically distributed monitoring system based on hemodynamics, to monitor the human arterial system by real-time acquisition of pulse signals, an electrocardiogram signal and a phonocardiogram signal, extraction of characteristic information, and calculation of hemodynamic parameters.

Technical scheme: the present invention provides a construction method of a digital human cardiovascular system based on hemodynamics, which comprises the following steps:
(1) partitioning various parts of a human body according to a distributed hemodynamic monitoring system, and determining granularity of a digital human model system;
(2) constructing an arterial blood flow transmission equation based on an elastic tube model for arteries, and defining physiological meanings for characteristic values of the equation;
(3) inputting signals, characteristic values and hemodynamic parameters acquired by the distributed hemodynamic monitoring system into the digital human model system; and
(4) obtaining a dynamic and distributed digital human cardiovascular system through finite element calculation.

The distributed hemodynamic monitoring system comprises a pulse sensor, an electrocardiogram sensor, a phonocardiogram sensor and a signal acquisition and analysis system; the pulse sensor comprises arterial pulse sensors arranged at superficial arteries of the human body and photoplethysmography sensors arranged at capillaries of the human body; the electrocardiogram sensor is used for acquiring electrocardiogram signals under different leads; the phonocardiogram sensor is used for acquiring phonocardiogram signals at different parts; and the signal acquisition and analysis system is connected with the sensors and dynamically receives detected physiological signals of the human body in real time, and performs analysis of the signals, extraction of the characteristic values, calculation of the hemodynamic parameters, and storage of the signals, the characteristic values and the parameters.

The distributed hemodynamics monitoring system processes the acquired signals, the signal processing comprising the following steps:
(1) preprocessing of pulse, electrocardiogram and phonocardiogram signals, for improving the signal-to-noise ratio of the signals and eliminating interference signals;
(2) extraction of characteristic values of various signals, including intensity difference and time difference among characteristic points;
(3) multi-signal cooperative analysis, for extracting characteristic values among signals based on synchronous acquisition of the signals; and
(4) calculation of hemodynamic parameters, to obtain various cardiovascular parameters according to the pulse, electrocardiogram and phonocardiogram signals and preset information.

Preferably, the arterial blood flow transmission equation is:

$$- \frac{\partial \text{P}}{\partial \text{x}} = L \frac{\partial Q}{\partial t} + R Q$$

$$- \frac{\partial \text{Q}}{\partial \text{x}} = C \frac{\partial P}{\partial t} ;$$

wherein P and Q represents the average pressure and flow along the cross-section of the vessels, respectively; x represents a coordinate and t represents time; and L, R and C represents flow inertia, viscous resistance, and vessel wall compliance, respectively.

Furthermore, in the arteries, the flow inertia

$$\text{L} \text{=} \frac{9 ρ}{4 π \text{} {r}_{0}^{2}} l ,$$

the viscous resistance

$$\text{R} \text{=} \frac{81 μ}{8 π \text{} {r}_{0}^{4}} l$$

and the vessel wall compliance

$$\text{C} = \frac{2 π {r}_{0}^{3}}{2 E h} l ;$$

wherein ρ, µ, r₀, l, E and h represents blood density, blood viscosity coefficient, untensioned vessel radius, length of a vessel segment, Young’s modulus of vessel wall, and thickness of vessel wall, respectively.

The human cardiovascular parameters monitored by the distributed hemodynamic monitoring system include: an electrocardiogram signal under standard limb leads, a phonocardiogram signal at aorta, pulse signals at left and right radial arteries, pulse signals at left and right brachial arteries, pulse signals at left and right instep arteries, pulse signals at left and right tibial arteries and a pulse signal at a common carotid artery.

In the step (1), the preprocessing comprises Fourier filtering, wavelet transformation, adaptive filtering, and a mathematical morphology method.

In the step (3), the multi-signal cooperative analysis is based on the synchronous acquisition of the pulse, electrocardiogram and phonocardiogram signals, and the characteristic values among the signals comprise cardiovascular health information along the pulse wave transmission route; and the characteristic values include: pulse arrival time PAT, pulse transmit time PTT, pre-ejection period PEP and pulse wave velocity PWV, wherein PAT = PEP + PTT.

The present invention also provides a digital human cardiovascular system constructed by the above method.

The present invention also provides an application of the digital human cardiovascular system based on hemodynamics in non-disease diagnosis, such as disease research, medical training, drug development, the telemedical technology combined with augmented reality/virtual reality AR/VR.

(1) The prediction of diagnosis of human cardiovascular health state: based on the user’s individual digital human model and the hemodynamic principle, the evolution process of the user’s cardiovascular state, including the evolution process of existing diseases and the probability of other diseases, are calculated and estimated, and medical advice is provided.

(2) The disease research: the characteristics of the digital human model corresponding to the disease are counted through a big data technology, and the evolution process of the disease is further calculated and estimated. The digital human model can dynamically present the load (such as blood pressure) of various parts of the human body, which has a positive effect on the disease mechanism research.

(3) The medical training: the human cardiovascular system and the evolution process of related diseases can be visually displayed by using a distributed and dynamic digital human model and abundant samples based on big data, which plays a positive role in the theoretical and professional training for related specialists.

(4) The drug development simulation: drug effects can be digitalized and loaded on the digital human model by pharmaceutical companies, to calculate and simulate the changes in the human cardiovascular state and the evolution process of diseases, which plays a positive role in the drug research and development and research on medication time and position.

(5) The telemedical technology: the digital human model can be combined with 5G technology and AR/VR technology, to achieve the telemedicine. The digital human model of a user can be dynamically displayed in real time in front of a doctor, so that the quality of diagnosis and treatment is improved. The digital human model has a promoting effect on the realization of the telemedicine and plays a positive role in the future medical model and the redistribution of medical resources.

Furthermore, the pulse sensor of the present invention has a number of 2 or more, and is classified into two types, namely an arterial pulse wave sensor and a photoplethysmography sensor. The arterial pulse wave sensor can be classified into a mechanical sensor for sensing pressure and strain and an ultrasonic sensor based on an ultrasonic principle. The arterial pulse sensors are arranged at superficial arteries of the human body (such as a common carotid artery, an external carotid artery, a brachial artery, a radial artery, a tibial artery and an instep artery) in a distributed manner. The photoplethysmography sensors are arranged at capillaries of the human body (such as finger tips and earlobes) in a distributed manner.

The electrocardiogram sensor has 1 or more lead modes, in which the electrodes are arranged at four limbs, the front part of the chest and the back part of the chest in a distributed manner. Various lead modes such as unipolar leads, bipolar leads, and chest leads can be constructed as required, and electrocardiogram signals under different leads are acquired.

The phonocardiogram sensor has a number of 1 or more, and can be arranged at aorta, lung, tricuspid valve and mitral valve as required to acquire phonocardiogram signals at different parts.

The signal acquisition and analysis system comprises the following functions: acquisition of pulse/electrocardiogram/phonocardiogram signals, storage of the signals, processing of the signals, extraction of characteristic values of the signals, calculation of hemodynamic parameters, human-machine interaction (display and voice), and communication (with a mobile phone, a computer and other terminals and a cloud).

Based on a systemically distributed monitoring system based on hemodynamics, the present invention provides a corresponding signal processing algorithm and constructs a digital human cardiovascular model system based on hemodynamics.

The signal processing algorithm comprises a preprocessing algorithm of pulse, electrocardiogram and phonocardiogram signals, an extraction algorithm of characteristic values of the various signals, a multi-signal collaborative analysis algorithm and a calculation algorithm of hemodynamic parameters, and the specific algorithms and models are as follows.

(1) The preprocessing algorithm of pulse, electrocardiogram and phonocardiogram signals: comprises Fourier filtering, wavelet transformation, adaptive filtering and a mathematical morphology method, and is used for improving the signal-to-noise ratio of the signals and eliminating interference signals.

(2) The extraction algorithm of characteristic values: the extracted characteristic values include: systolic peak intensity and time, tidal wave intensity and time, diastolic peak intensity and time, trough intensity and time, starting point intensity and time, systolic peak slope, diastolic slope, systolic waveform area, diastolic waveform area, systolic duration, diastolic duration, period, and intensity difference and time difference among characteristic points of pulse signals; intensity and time of P wave, QRS wave, T wave and U wave, and intensity difference and time difference among characteristic points of the electrocardiogram signal; intensity and time of a first phonocardiogram peak, time of a first phonocardiogram starting point, intensity and time of a second phonocardiogram peak, time of a second phonocardiogram starting point, duration of a first phonocardiogram time, duration of a second phonocardiogram time, period and intensity difference and time difference among characteristic points of the phonocardiogram signal. Moreover, benefiting from the characteristic of distributed arrangement of the sensors, the characteristic values of the pulse signals have the property of spatial distribution.

(3) The signal collaborative analysis algorithm: aims at proposing characteristic values among signals on the basis of synchronous acquisition of the signals, which include: pulse arrival time, pulse transmit time, pre-ejection period, and pulse wave velocity. Those characteristic values are calculated in different ways according to different characteristic points for pulse, electrocardiogram and phonocardiogram signals. Moreover, benefiting from the characteristic of distributed arrangement of the sensors of the patent, the pulse wave velocity has the property of spatial distribution.

(4) The calculation algorithm of hemodynamic parameters: aims at obtaining various cardiovascular parameters according to the pulse, electrocardiogram and phonocardiogram signals and preset information. The preset information includes, as required: age, sex, height, body weight, body fat percentage, and blood viscosity. The cardiovascular parameters include: heart rate, oxyhemoglobin saturation, systolic pressure, diastolic pressure, waveform characteristic quantity, mean arterial pressure, stroke volume, cardiac output, stroke volume index, cardiac index, cardiac function index, total peripheral resistance, arterial compliance, half-update rate of blood flow, half-update time of blood flow, mean residence time of blood flow, and total blood volume. Furthermore, respiration rate can be demodulated at the same time. Moreover, benefiting from the characteristic of distributed arrangement of the sensors of the patent, the related hemodynamic parameters have the property of spatial distribution.

The technical difficulties of the present invention are as follows.

(1) The distributed hemodynamic monitoring system needs to have high detection density and high throughput signal acquisition capability to dynamically monitor the state of the whole cardiovascular system of a human body in real time, so that high spatial granularity and time resolution of the digital human cardiovascular system are guaranteed.

(2) The digital human model provided by the present invention needs to have abundant and various signal analysis technologies to achieve accurate and comprehensive construction of the physiological digital body model.

### Beneficial Effects

the hemodynamic monitoring system provided by the present invention inherits the characteristics of real-time dynamics in time and simplicity and portability in use and experience of non-invasive hemodynamic monitoring technology, and achieves the advantage of systemic distributed measurement in space, laying a hardware foundation for the construction of the digital human cardiovascular system model.

According to a systemically distributed monitoring system based on hemodynamics, the present invention provides a corresponding signal processing algorithm and constructs a digital human cardiovascular system model based on hemodynamics.

The digital human model provided by the present invention can visually present cardiovascular parameters of various parts of a human body, and dynamically display the blood pressure and the state of blood flow of arteries of various parts of the human body and the electromechanical behavior of the heart.

According to the system constructed by the present invention, an individual dynamic digital human model based on hemodynamics is constructed through the distributed characteristic values of various parts of the human body and the calculated hemodynamic parameters. The model can dynamically record and display cardiovascular states of various parts of the human body in real time, and thus is of great medical value.

### BRIEF DESCRIPTION OF THE SEVERAL VIEWS OF THE DRAWINGS

FIG. 1 shows a schematic diagram of a human body wearing a systemically distributed monitoring system based on hemodynamics.

FIG. 2 shows a pulse signal acquired by the system and a schematic diagram of characteristic values of the pulse signal, wherein panel (a) shows the pulse signal, and panel (b) shows a schematic diagram of the characteristic values of the pulse signal.

FIG. 3 shows an electrocardiogram signal acquired by the system and a schematic diagram of characteristic values of the electrocardiogram signal, wherein panel (a) shows the electrocardiogram signal acquired by the system, and panel (b) shows a schematic diagram of the characteristic values of the electrocardiogram signal.

FIG. 4 shows an original phonocardiogram signal acquired by the system, a respiration rate signal, a phonocardiogram signal and a schematic diagram of characteristic values of the phonocardiogram signal, wherein panel (a) shows the original phonocardiogram signal acquired by the system, panel (b) shows the respiration rate signal obtained based on the original phonocardiogram signal, panel (c) shows the processed phonocardiogram signal, and panel (d) shows a schematic diagram of the characteristic values of the phonocardiogram signal.

FIG. 5 shows a schematic diagram of the characteristic values of collaborative analysis of an electrocardiogram signal, a signal of envelope of a phonocardiogram signal, a pulse signal at a brachial artery and a pulse signal at a radial artery.

FIG. 6 shows a flowchart of the calculation of hemodynamic parameters.

FIG. 7 shows a schematic diagram of a digital human model.

FIG. 8 shows a schematic diagram of the principle of intelligent diagnosis of human diseases.

FIG. 9 shows a schematic diagram of a simulation process of drug research and development based on a digital human model.

### DETAILED DESCRIPTION OF THE INVENTION

The present invention will be further described in detail below with reference to the embodiments.

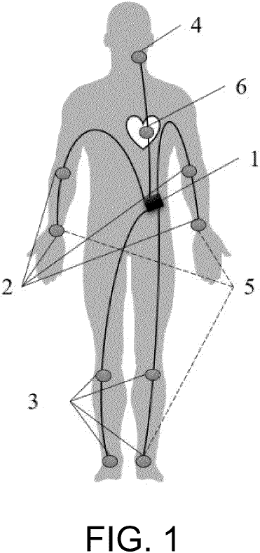

As shown in FIG. 1, a systemically distributed monitoring system based on hemodynamics generally comprises the following components: a signal acquisition and analysis system 1, upper limb pulse sensors 2, lower limb pulse sensors 3, a neck pulse sensor 4, an electrocardiogram sensor 5 and a phonocardiogram sensor 6.

The signal acquisition and analysis system 1 dynamically receives physiological signals of a human body as detected by the pulse sensors (the upper limb pulse sensors 2, the lower limb pulse sensors 3 and the neck pulse sensor 4), the electrocardiogram sensor 5 and the phonocardiogram sensor 6 in real time, and performs analysis of signals, extraction of characteristic values, calculation of hemodynamic parameters, and storage of signals, characteristic values and parameters. In addition, the system has a human-computer interaction capability, can display the physiological signals of the human body and inform the physiological state of the human body by displaying images and voice and other ways and can be controlled to a certain extent by a user. The system further has the function of communicating with terminals such as a mobile phone and a computer, and enables the displaying and storage of data on various terminals in real time through wireless communication technologies such as Bluetooth and WIFI. The system can transmit data to a cloud server through the Internet to establish a big data sample library for the artificial intelligence technology to learn.

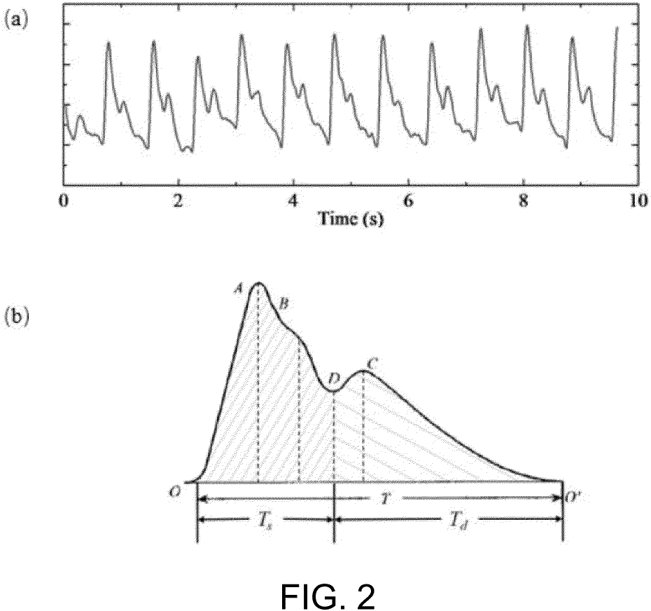

The pulse sensors (the upper limb pulse sensors 2, the lower limb pulse sensors 3 and the neck pulse sensor 4) can be arranged at superficial arteries or capillaries as required to acquire pulse wave signals. In this embodiment, the upper limb pulse sensors 2 are arranged at left and right radial arteries and left and right brachial arteries, the lower limb pulse sensors 3 are arranged at left and right instep arteries and left and right tibial arteries, and the neck pulse sensor is arranged at a common carotid artery. The pulse wave signals are acquired and stored by the signal acquisition and analysis system 1. The pulse wave signals are filtered by low-pass filtering at 15 Hz to remove high-frequency noise, and baseline drift is corrected by a mathematical morphology method. (a) of FIG. 2 shows the processed pulse wave signal at radial arteries, and the schematic diagram of details is shown in (b) of FIG. 2. The waveform clearly shows the starting point O, the systolic peak A, the tidal wave B, the diastolic peak C and the trough D of the pulse wave. The characteristic values described in Summary are extracted by a mathematical morphology method and a dynamic threshold method. With the distributed pulse sensors, the respective characteristic values of the arteries of the limbs and the aorta can be extracted, and the hemodynamic parameters can be calculated. The difference of the characteristic values and parameters provides abundant distributed information for diagnosis and treatment of cardiovascular diseases.

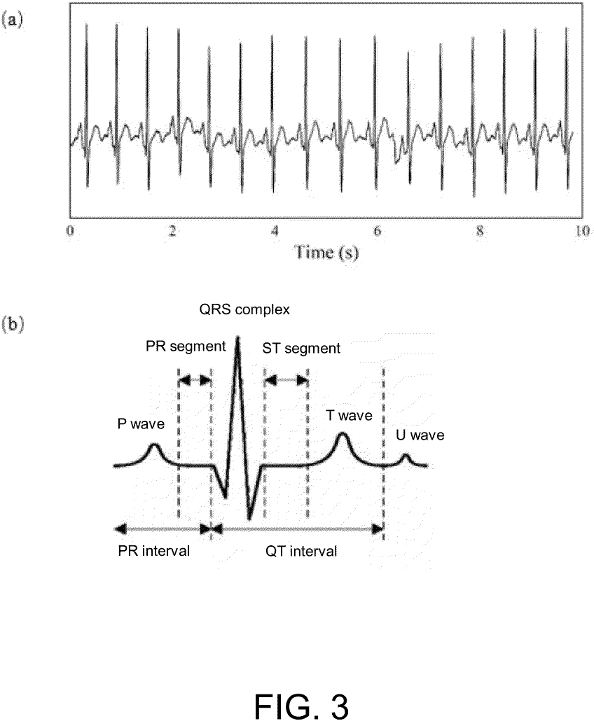

The electrocardiogram sensors 5 can be used to construct limb leads or chest leads. In this embodiment, the electrocardiogram electrodes are arranged at left and right wrists and one ankle, to construct standard limb leads. The electrocardiogram signals are acquired and stored by the signal acquisition and analysis system 1. The electrocardiogram signals are filtered by low-pass filtering at 100 Hz to remove high-frequency noise, and baseline drift is corrected by a mathematical morphology method. (a) of FIG. 3 shows the electrocardiogram signal under the limb leads acquired by the system, and the schematic diagram of details is shown in (b) of FIG. 3. The waveform clearly shows the P wave, QRS complex, T wave and U wave of the electrocardiogram signal. The characteristic values described in Summary are extracted by a mathematical morphology method and a dynamic threshold method.

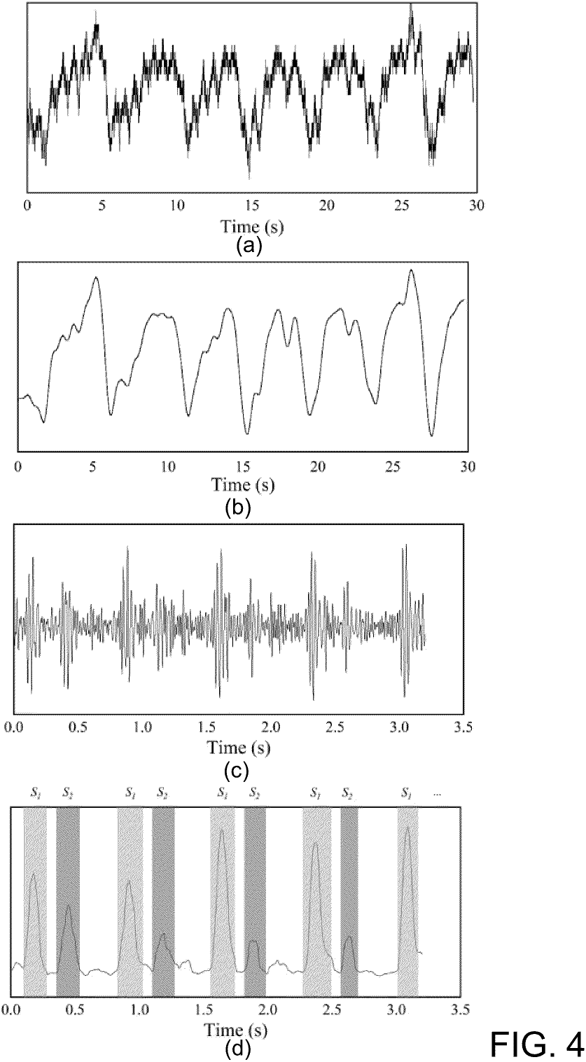

The phonocardiogram sensors 6 can be arranged at aorta, lung, tricuspid valve, and mitral valve as required to acquire phonocardiogram signals at different parts. In this embodiment, the phonocardiogram sensor is arranged at aorta. The phonocardiogram signals are acquired and stored by the signal acquisition and analysis system 1. (a) of FIG. 4 shows an original phonocardiogram signal acquired by the system. The envelope of the phonocardiogram signal contains information about the expansion of the chest cavity due to respiration. As shown in (b) of FIG. 4, the respiration signal is extracted by low-pass filtering at 3 Hz and used for the calculation of the respiration rate. The phonocardiogram signal is extracted by band-pass filtering of the original phonocardiogram signal at 20-100 Hz, as shown in (c) of FIG. 4. The first and second phonocardiogram events can be clearly observed. The envelope of the phonocardiogram signal is extracted by a normalized Shannon energy method and a mathematical morphology method (see (d) of FIG. 4). Further, the characteristic values described in Summary are extracted by a mathematical morphology method and a dynamic threshold method.

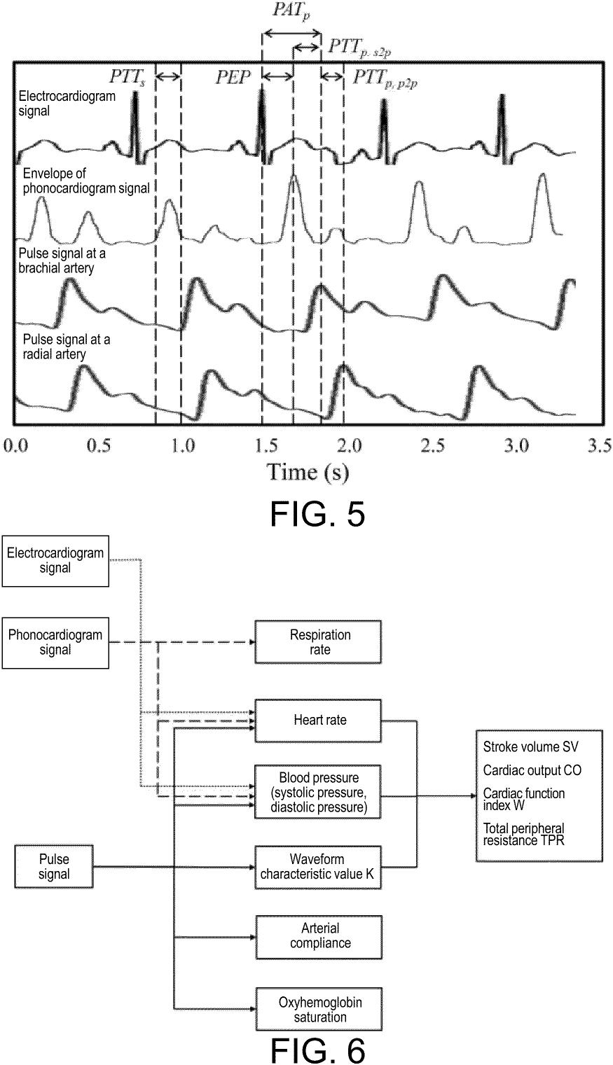

FIG. 5 shows a multi-signal collaborative analysis algorithm. In FIG. 5, an electrocardiogram signal, a signal of envelope of a phonocardiogram signal, a pulse signal at a brachial artery and a pulse signal at a radial artery are synchronously acquired, and the above signals are cooperatively analyzed to obtain characteristic values, so as to demodulate cardiovascular health information along a pulse wave transmission route. The characteristic values include pulse arrival time PAT, pulse transmit time PTT, pre-ejection period PEP, and pulse wave velocity PWV. The pulse arrival time refers to the time difference between the generation of an electrical signal by the heart and the detection of a pulse wave at the distal end. The pulse transmit time refers to the time difference between the ejection by the heart and the detection of a pulse wave at the distal end. The pre-ejection period refers to the time difference between the generation of an electrical signal by the heart and the ejection by the heart. Therefore, the three characteristic values generally have the following relationship: PAT = PEP + PTT.

The pulse wave velocity refers to the velocity at which a pulse wave is transmitted in arteries. Benefiting from the characteristic of distributed arrangement of the sensors, the pulse wave velocities of different arteries of a human body can be calculated separately. In this embodiment, the calculation of the pulse wave velocities of the aorta segment, the heart-brachial artery segment, the radial artery segment, the heart-tibial artery segment, and the tibia-instep artery segment is implemented. Those characteristic values are calculated in different ways according to different characteristic points for pulse, electrocardiogram and phonocardiogram signals. In this embodiment, the pulse arrival time PATp, the pulse transmit time PTTp, and the pre-ejection period PEP are calculated from the R-wave peak of the electrocardiogram signal, the S1 phonocardiogram peak of the phonocardiogram signal, and the systolic peak of the pulse wave signal. The related characteristic values can also be calculated from characteristic points such as a starting point and a middle point of the signal.

The hemodynamic parameters can be derived from the pulse 2/3/4, electrocardiogram 5, and phonocardiogram 6 signals. FIG. 6 shows a flow chart of the calculation of human physiological parameters. The respiration rate RR is derived from the envelope of the phonocardiogram 6 signal. The heart rate HR is related to the period T of the pulse 2/3/4, electrocardiogram 5 and phonocardiogram 6 signals. The blood pressure including the systolic pressure Ps and the diastolic pressure Pd is strongly correlated with the pulse arrival time PAT, the pulse transmit time PTT, the pre-ejection period PEP, the pulse wave velocity PWV, and the pulse waveform. In this embodiment, the blood pressure is calculated by the formula BP = a × PWV + b. The oxyhemoglobin saturation is calculated from the alternating current and direct current components of the photoplethysmography pulse wave. The formulas for calculating other hemodynamic parameters are listed below:

| | |
|---|---|
| Waveform characteristic value K | K = (Pₘ - P_{d})/(Pₛ - P_{d}) |
| Arterial compliance AC | AC = 0.283 T/K² |
| Stroke volume SV | SV = 0.283T(Pₛ - P_{d})/K² |
| Cardiac output CO | CO = 17(Pₛ - P_{d})/K² |
| Cardiac function index W | W = T(Pₛ - P_{d}) |
| Total peripheral resistance TPR | TPR = Pₘ/CO |

It should be understood that the formulas are only a specific embodiment of the present invention, and are not intended to limit the present invention.

In this embodiment, the hemodynamic parameters of a 26-year-old male with a healthy cardiovascular system acquired by the system are listed below:

| | |
|---|---|
| Heart rate | 78 |
| Systolic pressure Pₛ | 137 mmHg |
| Diastolic pressure P_{d} | 80 mmHg |
| Waveform characteristic value K | 0.38 |
| Arterial compliance AC | 1.51 mL/mmHg |
| Stroke volume SV | 85.9 mL/beat |
| Cardiac output CO | 6.7 L/min |
| Cardiac function index W | 0.73 |
| Total peripheral resistance TPR | 0.466 PRU |

In this embodiment, the hemodynamic parameters of a 26-year-old male with a healthy cardiovascular system acquired by the system are listed below:

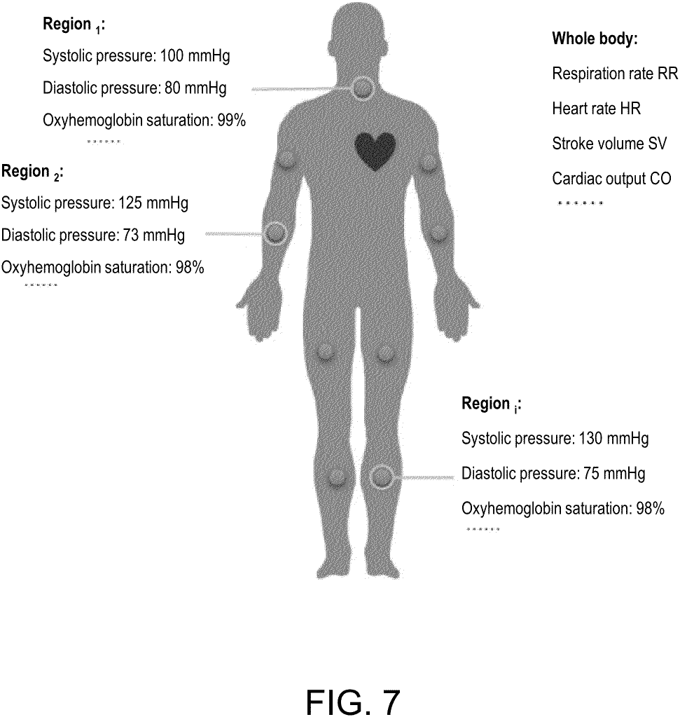

Benefiting from the characteristic of the systemic distribution of the hemodynamic system, the pulse 2/3/4 signals, the pulse wave velocity PWV and the related hemodynamic parameters (such as blood pressure, oxyhemoglobin saturation, waveform characteristic value K, and arterial compliance) have spatially distributed differences. Thus, a health monitoring system for the human arterial system is constructed. As shown in FIG. 7, in this embodiment, the distributed monitoring of the aorta segment, the left and right heart-brachial artery segments, the left and right radial artery segments, the left and right heart-tibial artery segments, and the left and right tibial-instep artery segments is implemented. In addition, the more accurate measurement of parameters such as respiration rate RR, heart rate HR, stroke volume SV and cardiac output CO is achieved by weighting average of the characteristic values of signals of various parts.

Some of the hemodynamic-related parameters at different parts of a 26-year-old male with a healthy cardiovascular system are listed below:

| | | | |
|---|---|---|---|
| Part | Pulse arrival/ transmit time | Systolic pressure | Diastolic pressure |
| Aorta segment | 215.5 ms | 132 mmHg | 75 mmHg |
| Left heart-brachial artery segment | 253.3 ms | 120 mmHg | 78 mmHg |
| Right heart-brachial artery segment | 256.7 ms | 126 mmHg | 78 mmHg |
| Left radial artery segment | 29.0 ms | 121 mmHg | 80 mmHg |
| Right radial artery segment | 28.2 ms | 125 mmHg | 79 mmHg |
| Left heart-tibial artery segment | 329.3 ms | 150 mmHg | 101 mmHg |
| Right heart-tibial artery segment | 318.7 ms | 154 mmHg | 99 mmHg |

The pulse arrival time, i.e., the time difference between the generation of an electrocardiogram signal by the heart and the detection of the pulse wave at the distal end, is calculated in the aorta segment, the left heart-brachial artery segment, the right heart-brachial artery segment, the left heart-tibial artery segment and the right heart-tibial artery segment; the pulse transmit time, i.e., the time difference of the pulse wave being transmitted from the proximal end to the distal end, is calculated in the left radial artery segment and the right radial artery segment.

An individual dynamic digital human model based on hemodynamics is constructed through the distributed characteristic values of various parts of the human body and the calculated hemodynamic parameters. The model can dynamically record and display cardiovascular states of various parts of the human body in real time, and thus is of great medical value.

The digital human model described in the patent is constructed in the following specific steps:
step 1: partitioning various parts of a human body according to a distributed hemodynamic monitoring system, and determining granularity of a digital human model;
step 2: constructing an arterial blood flow transmission equation based on an elastic tube model for arteries, and defining physiological meanings for characteristic values of the equation;
step 3: inputting signals, characteristic values and hemodynamic parameters acquired by the distributed hemodynamic monitoring system into the digital human model; and
step 4: obtaining a dynamic and distributed cardiovascular system digital human model through finite element calculation.

In this embodiment, the human cardiovascular parameters monitored by the distributed hemodynamic monitoring system include: an electrocardiogram signal under standard limb leads, a phonocardiogram signal at aorta, pulse signals at left and right radial arteries, pulse signals at left and right brachial arteries, pulse signals at left and right instep arteries, pulse signals at left and right tibial arteries and a pulse signal at a common carotid artery. Therefore, the constructed digital human model can be divided into an aorta segment, a heart-brachial artery segment, a radial artery segment, a heart-tibial artery segment and a tibia-instep artery segment.

In this embodiment, the arterial blood flow transmission equation is:

$$- \frac{\partial \text{P}}{\partial \text{x}} = L \frac{\partial Q}{\partial t} + R Q$$

$$- \frac{\partial \text{Q}}{\partial \text{x}} = C \frac{\partial P}{\partial t}$$

wherein P and Q represents the average pressure and flow along the cross-section of the vessels, respectively; x represents a coordinate and t represents time; and L, R and C represents flow inertia, viscous resistance, and vessel wall compliance, respectively.

In the arteries, the flow inertia

$$\text{L=} \frac{9 ρ}{4 π {r}_{0}^{2}} l ,$$

the viscous resistance

$$\text{R} \text{=} \frac{81 μ}{8 π \text{} {r}_{0}^{4}} l ,$$

and the vessel wall compliance

$$\text{C} = \frac{3 π {r}_{0}^{3}}{2 E h} l ,$$

wherein ρ, µ r₀, l, E and h represents blood density, blood viscosity coefficient, untensioned vessel radius, length of a vessel segment, Young’s modulus of vessel wall, and thickness of vessel wall, respectively.

Preset signals (such as age, sex, height and body weight) enable the length of each vessel segment to be estimated. In this embodiment, for a 26-year-old male with a healthy cardiovascular system in a height of 178 cm, the length of the blood vessel of the aorta segment is estimated to be 20 cm, the length of the blood vessel of the heart-brachial artery segment is estimated to be 45 cm, the length of the blood vessel of the radial artery segment is estimated to be 23 cm, the length of the blood vessel of the heart-tibial artery segment is estimated to be 105 cm, and the length of the blood vessel of the tibia-instep artery segment is estimated to be 33 cm.

The level I information (pulse 2/3/4, electrocardiogram 5, a phonocardiogram waveform signal) acquired by the system reflects the pressure and flow of each artery segment and the transmission time of the pressure and flow in the blood vessel. In this embodiment, the pressure and transmission time of each artery segment are listed in the table above.

The arterial compliance and total peripheral resistance of each artery segment can be calculated from the level II information (hemodynamic parameters) by the system. The calculation method is as described above.

Based on the above three levels of information, the flow inertia, viscous resistance and vessel wall compliance of the aorta segment, the heart-brachial artery segment, the radial artery segment, the heart-tibial artery segment and the tibia-instep artery segment can be calculated through an arterial blood flow transmission equation, and then physiological parameters such as blood density, blood viscosity coefficient, Young’s modulus of vessel wall, and thickness of vessel wall can be calculated.

Finally, the division of the human cardiovascular system is refined through the arterial blood flow transmission equation to reduce the granularity of the digital human model, and obtain a human cardiovascular system model with higher spatial distribution rate.

The human cardiovascular system model has the following functions and use: the precise medical technology for intelligent diagnosis of human diseases; prediction of human cardiovascular health state based on hemodynamics; disease mechanism research; medical teaching and training; drug research and development simulation; and the telemedical technology combined with augmented reality/virtual reality AR/VR.

In this embodiment, the precise medical technology for intelligent diagnosis of human diseases and drug research and development simulation technology are described in detail.

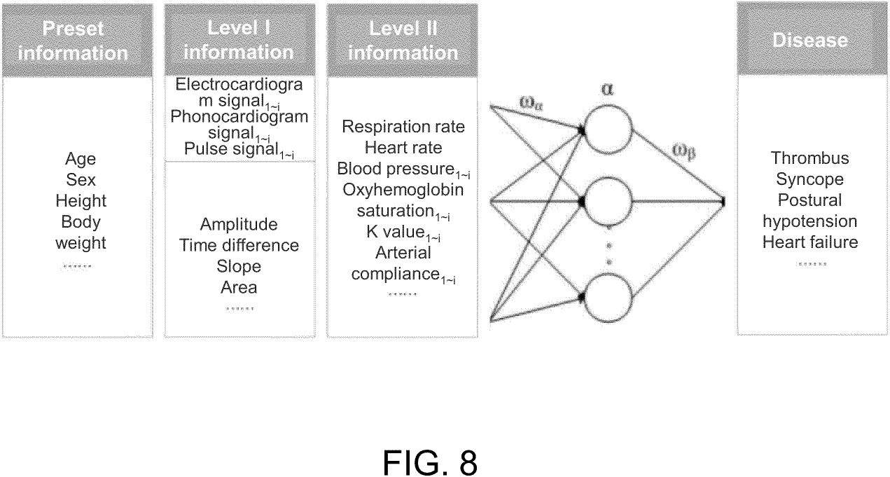

As shown in FIG. 8, after the user presets basic information such as age, sex, height and body weight and uses the system to acquire the level I information (pulse 2/3/4, electrocardiogram 5 and phonocardiogram waveform signals) and the calculated level II information (hemodynamic parameters), the system can comprehensively diagnose related diseases by a correlation matrix of the diseases and the signals and a neural network technology. Benefiting from the distributed hemodynamic monitoring system, the corresponding level I information and level II information comprise human body distribution information, which greatly improves the accuracy of the diagnosis of related diseases, for example, diagnosis of thrombus, determination of lesion position, diagnosis of postural hypotension, and tracing of systemic etiology of heart failure.

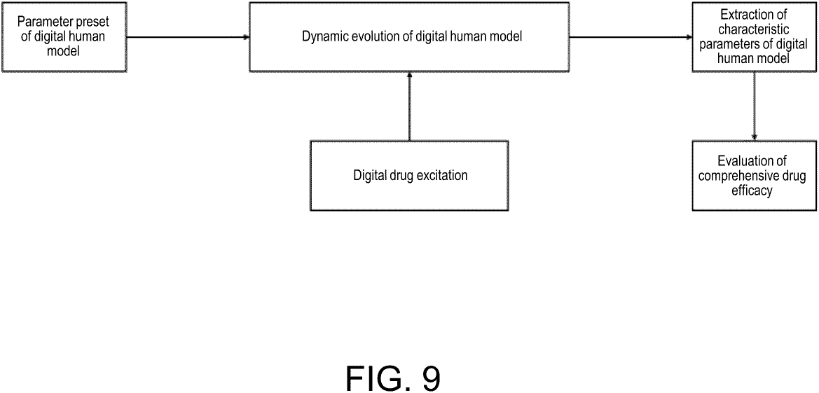

As shown in FIG. 9, in the processes of research and development of drugs and medical devices, an enterprise can firstly construct a digital human model for a corresponding disease. In the dynamic evolution process of the digital human model, the drugs and medical devices with digital actions act on the digital human model as an external excitation, and the digital human model further provides feedbacks on the excitation. The enterprise can set relevant characteristic parameters for the human model to evaluate the utility of the drugs or medical devices. In addition, the enterprise can list relevant parameters of the drugs or the medical devices and define an evaluation function of the digital human model to quickly obtain the optimal properties of products. The digital human model is used as a safe, efficient, rapid and easy-to-use research and development simulation tool, and plays a positive role in promoting the research on the dosage and timing of the drugs or the medical devices.

1. A construction method of a digital human cardiovascular system based on hemodynamics, comprising the following steps:
(1) partitioning various parts of a human body according to a distributed hemodynamic monitoring system, and determining granularity of a digital human model system;
(2) constructing an arterial blood flow transmission equation based on an elastic tube model for arteries, and defining physiological meanings for characteristic values of the arterial blood flow transmission equation;
(3) inputting signals, characteristic values and hemodynamic parameters acquired by the distributed hemodynamic monitoring system into the digital human model system; and
(4) obtaining a dynamic and distributed digital human cardiovascular system through a finite element calculation.

2. The construction method of the digital human cardiovascular system based on hemodynamics according to claim 1, wherein the distributed hemodynamic monitoring system comprises a pulse sensor, an electrocardiogram sensor, a phonocardiogram sensor and a signal acquisition and an analysis system; the pulse sensor comprises arterial pulse sensors arranged at superficial arteries of the human body and photoplethysmography sensors arranged at capillaries of the human body; the electrocardiogram sensor is used for acquiring electrocardiogram signals under different leads; the phonocardiogram sensor is used for acquiring phonocardiogram signals at different parts; and the signal acquisition and the analysis system is connected with sensors and dynamically receives detected physiological signals of the human body in real time, and performs analysis of the signals, extraction of the characteristic values, calculation of the hemodynamic parameters, and storage of the signals, the characteristic values and the parameters.

3. The construction method of the digital human cardiovascular system based on hemodynamics according to claim 1, wherein the distributed hemodynamic monitoring system processes acquired signals, which are acquired, the signal processing comprising the following steps:
(1) preprocessing of pulse, electrocardiogram and phonocardiogram signals, for improving a signal-to-noise ratio of the signals and eliminating interference signals;
(2) extraction of characteristic values of various signals, including intensity difference and time difference among characteristic points;
(3) multi-signal cooperative analysis, for extracting characteristic values among the signals based on a synchronous acquisition of the signals; and
(4) calculation of the hemodynamic parameters, to obtain various cardiovascular parameters according to the pulse, electrocardiogram and the phonocardiogram signals and preset information.

4. The construction method of the digital human cardiovascular system based on hemodynamics according to claim 1, wherein the arterial blood flow transmission equation is:
$$- \frac{\partial \text{P}}{\partial \text{x}} = L \frac{\partial Q}{\partial t} + R Q$$
$$- \frac{\partial \text{Q}}{\partial \text{x}} = C \frac{\partial P}{\partial t} ;$$
wherein P and Q represents an average pressure and a flow along a cross-section of vessels, respectively; x represents a coordinate and t represents time; and L, R and C represents a flow inertia, a viscous resistance, and a vessel wall compliance, respectively.

5. The construction method of the digital human cardiovascular system based on hemodynamics according to claim 4, wherein, in the arteries, the flow inertia
$$\text{L} = \frac{9 ρ}{4 π {r}_{0}^{2}} l ,$$
the viscous resistance
$$\text{R} = \frac{81 μ}{8 π {r}_{0}^{4}} l ,$$
and the vessel wall compliance
$$\text{C} = \frac{3 π {r}_{0}^{3}}{2 E h} l ;$$
wherein p, µ, r₀, l, E and h represents a blood density, a blood viscosity coefficient, an untensioned vessel radius, a length of a vessel segment, Young’s modulus of vessel wall, and a thickness of vessel wall, respectively.

6. The construction method of the digital human cardiovascular system based on hemodynamics according to claim 1, wherein human cardiovascular parameters monitored by the distributed hemodynamic monitoring system include: an electrocardiogram signal under standard limb leads, a phonocardiogram signal at an aorta, pulse signals at left and right radial arteries, pulse signals at left and right brachial arteries, pulse signals at left and right instep arteries, pulse signals at left and right tibial arteries and a pulse signal at a common carotid artery.

7. The construction method of the digital human cardiovascular system based on hemodynamics according to claim 1, wherein in the step (1), the preprocessing comprises Fourier filtering, wavelet transformation, adaptive filtering, and a mathematical morphology method.

8. The construction method of the digital human cardiovascular system based on hemodynamics according to claim 1, wherein in the step (3), a multi-signal cooperative analysis is based on a synchronous acquisition of a pulse, an electrocardiogram and phonocardiogram signals, and characteristic values among the signals comprise cardiovascular health information along a pulse wave transmission route; and the characteristic values include: a pulse arrival time PAT, a pulse transmit time PTT, a pre-ejection period PEP and a pulse wave velocity PWV, wherein PAT = PEP + PTT.

9. A digital human cardiovascular system constructed by the method according to claim 1.

10. Application of the digital human cardiovascular system according to claim 9 in non-disease diagnosis.
